(19) United States
(12) Patent Application Publication
(10) Pub. No.: US 2025/0107497 A1
(43) Pub. Date: Apr. 3, 2025

(54) APPARATUS AND METHODS FOR WOUNDING PLANT TISSUE

(71) Applicant: Pairwise Plants Services, Inc., Durham, NC (US)

(72) Inventors: Douglas Lee Heckart, Wake Forest, NC (US); Keith Alan Koutsky, Durham, NC (US)

(21) Appl. No.: 18/726,266

(22) PCT Filed: Jan. 6, 2023
(86) PCT No.: PCT/US2023/060193

§ 371 Date: Jul. 2, 2024

Related U.S. Application Data
(60) Provisional application No. 63/296,897, filed on Jan. 6, 2022.

(51) Int. Cl. A01H 4/00; C12N 15/82

(52) U.S. Cl. CPC A01H 4/003; A01H 4/002; C12N 15/8205

(57) ABSTRACT

Described herein are apparatus and methods for wounding plant tissue. A method of the present invention includes blending a plant tissue (e.g., a plant and/or plant part) in a blender (e.g., an electric blender) to provide wounded plant tissue.

### FIELD

The present invention relates to apparatus and methods for wounding plant tissue such as methods that include blending a plant tissue (e.g., a plant and/or plant part) in a blender to provide wounded plant tissue.

### BACKGROUND

In vitro plant propagation is a laborious process involving many tissue dissection and transfer steps that significantly limit throughput, increase duration of end to end process, and cause ergonomic stress on the practitioner. For a genetic modification pipeline (either transgenic or editing) where a large number of plant manipulations are required for success, the labor of tissue culture can be prohibitive. Accordingly, new approaches for preparing plant tissue are needed.

### SUMMARY OF THE INVENTION

One aspect of the present invention is directed to a method of preparing a plant tissue for propagation, regeneration, and/or transformation, the method comprising: wounding a plant or plant part to provide a wounded tissue (e.g., a cut tissue), wherein the wounding comprises blending the plant or plant part in a blender, thereby providing the plant tissue.

These and other aspects of the present invention are set forth in more detail in the description of the invention below.

### BRIEF DESCRIPTION OF THE FIGURES

FIG. 1 is an illustration of a chopping blade according to some embodiments of the invention.

FIG. 2 is another illustration of a chopping blade according to some embodiments of the invention.

### DETAILED DESCRIPTION

The present invention will now be described hereinafter with reference to the accompanying drawings and examples, in which embodiments of the invention are shown. This description is not intended to be a detailed catalog of all the different ways in which the invention may be implemented, or all the features that may be added to the instant invention.

For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

According to embodiments of the present invention provided are apparatus and methods for wounding plant tissue (e.g., a plant or plant part). In some embodiments, a method of the present invention comprises preparing a plant tissue for propagation, regeneration, and/or transformation. A method of the present invention may comprise wounding a plant or plant part to provide a wounded tissue (e.g., a cut tissue), wherein the wounding comprises blending the plant or plant part in a blender to thereby provide the wounded tissue. In some embodiments, the method includes culturing, propagating, regenerating, and/or transforming the wounded tissue. In some embodiments, the step of blending the plant or plant tissue in a blender is a step in a plant propagation, regeneration, and/or transformation method. In some embodiments, prior to wounding the plant or plant part, the method comprises collecting a plant part. For example, in some embodiments, a shoot grown from a node tissue may be collected and then blended in a blender to provided wounded plant tissue.

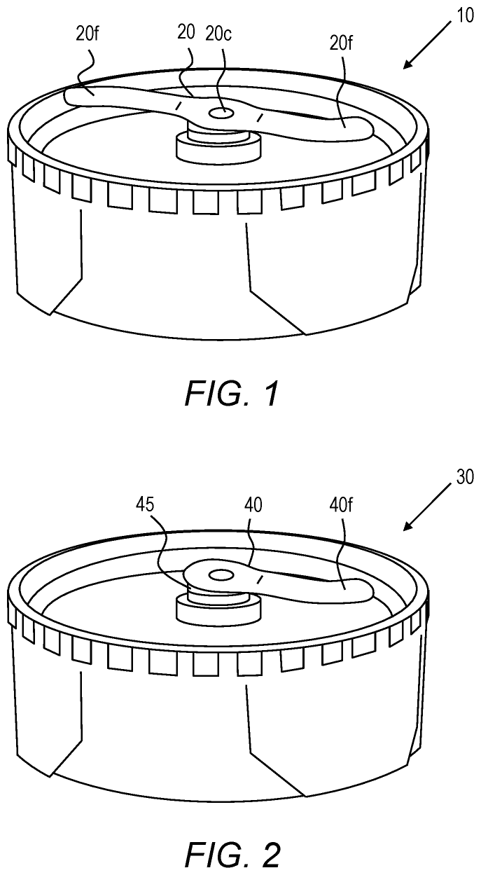

A “blender” as used herein refers to a mechanical device that is configured to blend, chop, dice, and/or slice material such as food. In some embodiments, the blender is powered by an electric motor (i.e., an electric blender). Exemplary electric blenders include, but are not limited to, a kitchen blender that includes a rotating blade (e.g., a metal blade such as a stainless steel blade) powered by an electric motor and a food processor that includes a blade (e.g., a cutting blade such as a metal (e.g., stainless steel) cutting blade) that is powered by an electric motor. The electric motor may be a 350-watt motor. In some embodiments, a blender used in a method of the present invention is a Cuisinart blender having model number CPB-300P1 or a blender comparable thereto and the blender may be fitted with a chopping blade. A chopping blade may also be referred to as an S blade. Exemplary chopping blades and/or S blades include, but are not limited to, a blade included in Cuisinart chopping assembly having model number CPB-300CHA or a blade comparable thereto. The blade may have a planar configuration. In some embodiments, the blade assembly 10 provided in a blender comprises a single elongate blade 20 that rotates about its center 20c such that it provides an edged “fin” 20f for chopping and/or cutting on either side of its pivot axis, as shown in FIG. 1. In some embodiments, a blade assembly 30 provided in a blender comprises a blade 40 with only a single edged “fin” 40f (see FIG. 2); the blade 40 rotates about a fixed axis 45 and employs only the single “fin” 40f for chopping and/or cutting. A blender of the present invention may be attached to a vessel operably associated with the blender such as Cuisinart cup having model number CTC-16. In some embodiments, the blender used in a method of the present invention is an electric blender (e.g., a kitchen blender and/or food processor) that includes and/or is fitted with a chopping blade and/or an S blade having metal (e.g., stainless steel) fins. One or more part(s) of a blender used in a method of the present invention may be able to be sterilized and/or configured for sterilization (e.g., alcohol sterilization and/or steam sterilization). In some embodiments, the parts of a blender that contact a plant and/or plant part may be sterilized and/or configured for sterilization. For example, a blender used in a method of the present invention may have a vessel (e.g., a cup), blade, and/or blade assembly that can be sterilized and/or that are configured for sterilization.

In a method of the present invention, the blender may be operated at a speed from about 1, 5, 10, 15, or 20 Hz to about 25, 30, 40, 50, 60, 70, 80, 90, 100, 150, 200, 250, 300, 350, or 380 Hz. In some embodiments, the blender may be set to and/or operated at the lowest speed setting for the blender, optionally set to and/or operated at a chop setting. In some embodiments, blending comprises blending plant material (e.g., a plant or plant part) at a speed from about 1, 5, 10, 15, or 20 Hz to about 25, 30, 35, 40, 45, or 50 Hz. The blending may be carried out for a period of time such as about 1, 2, 3, 4, 5, 6, 7, 8, 9, or 10 seconds or more, optionally using a pulse setting. In some embodiments, blending the plant material comprises blending the plant or plant part at one or more (e.g., 1, 2, 3, 4, 5, 6, 7, 8, 9, 10 or more) interval(s) with each interval including a time period during which the blender is operated to blend the plant material and optionally a time period during which the blender is not operated to thereby provide a rest from blending. In some embodiments, blending the plant material comprises one or more (e.g., 1, 2, 3, 4, 5, 6, 7, 8, 9, 10 or more) interval(s) with each interval including about 1 second to about 2, 3, 4, 5, 6, 7, 8, 9, or 10 seconds during which the blender is operated to blend the plant material and optionally about 1 second to about 2, 3, 4, 5, 6, 7, 8, 9, or 10 seconds during which the blender is not operated. In some embodiments, the blending step and/or the time to provide the wounded tissue is carried out in about 10 seconds or less such as about 9, 8, 7, 6, 5, 4, 3, 2, or 1 second(s). In some embodiments, the blending parameters and/or settings (e.g., speed, time, number of pulses, etc.) for the blender may depend on the plant tissue type and/or the purpose of the wounding (e.g., wounding for propagation, regeneration, or transformation).

“Blending,” “blend,” and grammatical variants thereof as used herein refer to mechanically cutting (e.g., chopping, dicing, etc.) material (e.g., plant material). Blending may cut the material into smaller pieces than the size of the material prior to blending. In some embodiments, blending may include coarsely cutting and/or finely cutting material. In some embodiments, blending is configured to coarsely cut plant material and aims to limit blending the plant material to a smoothie-like consistency. In some embodiments, blending provides cut plant material (e.g., wounded plant tissue) that has a size of about 0.2 or 0.3 cm to about 0.4 or 0.5 cm as measured at the widest part of the plant material. A plant or plant part may be blended in a blender. In some embodiments, blending comprises blending a whole plant or one or more part(s) thereof (e.g., an aerial portion and/or non-aerial portion of the plant). In some embodiments, blending comprises blending a leaf, node, shoot, and/or callus. In some embodiments, the blending of plant material is carried out in the presence of water and/or an aqueous composition (e.g., growth media). The plant material and water and/or aqueous composition may be added into a vessel of a blender and blended together. In some embodiments, plant material and bacteria, optionally in water and/or an aqueous composition, are blended together. The bacteria may be an Agrobacterium strain such as A. tumefaciens and/or A. rhizogenes. The bacteria may include a nucleic acid construct encoding all or a portion of an editing system (e.g., a CRISPR-Cas editing system) that is configured to modify a polynucleotide in a plant cell. Blending the plant material may inoculate the plant material with bacteria and/or introduce a polynucleotide from the bacteria into a cell of the plant material, optionally into a cell of the wounded tissue. Accordingly, a method of the present invention may comprise introducing a polynucleotide into a cell of the wounded plant tissue using the bacteria blended with plant tissue. In some embodiments, plant tissue is blended in water and/or an aqueous composition that is devoid of bacteria to provide the wounded plant tissue and the wounded plant tissue is contacted to bacteria, optionally to introduce a polynucleotide into a cell of the wounded plant tissue.

An “editing system” as used herein refers to any site-specific (e.g., sequence-specific) nucleic acid editing system now known or later developed, which system can introduce a modification (e.g., a mutation) in a nucleic acid in target specific manner. For example, an editing system (e.g., a site- and/or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which may comprise one or more polypeptide(s) and/or one or more polynucleotide(s) that when present and/or expressed together (e.g., as a system) in a composition and/or cell can modify (e.g., mutate) a target nucleic acid in a sequence specific manner. In some embodiments, an editing system (e.g., a site- and/or sequence-specific editing system) can comprise one or more polynucleotide(s) and/or one or more polypeptide(s), including but not limited to a nucleic acid binding polypeptide (e.g., a DNA binding domain), a nuclease, another polypeptide, and/or a polynucleotide.

In some embodiments, an editing system comprises one or more sequence-specific nucleic acid binding polypeptide(s) (e.g., a DNA binding domain) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system comprises one or more cleavage polypeptide(s) (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN).

A “nucleic acid binding polypeptide” as used herein refers to a polypeptide or domain that binds and/or is capable of binding a nucleic acid (e.g., a target nucleic acid). A DNA binding domain is an exemplary nucleic acid binding polypeptide and may be a site- and/or sequence-specific nucleic acid binding domain. In some embodiments, a nucleic acid binding polypeptide may be a sequence-specific nucleic acid binding polypeptide such as, but not limited to, a sequence-specific binding domain from, for example, a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein. In some embodiments, a nucleic acid binding polypeptide comprises a cleavage domain (e.g., a nuclease domain) such as, but not limited to, an endonuclease (e.g., Fok1), a polynucleotide-guided endonuclease, a CRISPR-Cas endonuclease, a zinc finger nuclease, and/or a transcription activator-like effector nuclease (TALEN). In some embodiments, the nucleic acid binding polypeptide associates with and/or is capable of associating with (e.g., forms a complex with) with one or more nucleic acid molecule(s) (e.g., forms a complex with a guide nucleic acid as described herein), which may direct and/or guide the nucleic acid binding polypeptide to a specific target nucleotide sequence (e.g., a gene locus of a genome) that is complementary to the one or more nucleic acid molecule(s) (or a portion or region thereof), thereby causing the nucleic acid binding polypeptide to bind to the nucleotide sequence at the specific target site. In some embodiments, the nucleic acid binding polypeptide is a CRISPR-Cas effector protein as described herein.

In some embodiments, an editing system comprises or is a ribonucleoprotein such as an assembled ribonucleoprotein complex (e.g., a ribonucleoprotein that comprises a CRISPR-Cas effector protein, a guide nucleic acid, and optionally a reverse transcriptase or a deaminase). In some embodiments, a ribonucleoprotein of an editing system may be assembled together (e.g., a pre-assembled ribonucleoprotein including a CRISPR-Cas effector protein, a guide nucleic acid, and optionally a reverse transcriptase or a deaminase) such as when contacted to a target nucleic acid or when introduced into a cell (e.g., a mammalian cell or a plant cell). In some embodiments, a ribonucleoprotein of an editing system may assemble into a complex (e.g., a covalently and/or non-covalently bound complex) while a portion of the ribonucleoprotein is contacting a target nucleic acid and/or may assemble after and/or during introduction into a plant cell. In some embodiments, an editing system may be assembled (e.g., into a covalently and/or non-covalently bound complex) when introduced into a plant cell. In some embodiments, a ribonucleoprotein may comprise a CRISPR-Cas effector protein, a guide nucleic acid, and optionally a reverse transcriptase. In some embodiments, a ribonucleoprotein may comprise a CRISPR-Cas effector protein, a guide nucleic acid, and optionally a deaminase.

In some embodiments, an editing system of the present invention comprises a reverse transcriptase, an extended guide nucleic acid, and a CRISPR-Cas effector protein, e.g., a Type II CRISPR-Cas effector protein or Type V CRISPR-Cas effector protein. In some embodiments, the Type V CRISPR-Cas effector protein or Type II CRISPR-Cas effector protein, the reverse transcriptase, and the extended guide nucleic acid may form a complex or may be comprised in a complex that is capable of interacting with a target nucleic acid.

In some embodiments, an editing system of the present invention is used in prime editing. “Prime editing” and grammatical variants thereof as used herein refer to a nucleic acid editing technology that uses a Cas9 nickase fused to a reverse transcriptase and modifies a target nucleic acid without a double strand break or a donor DNA template. In Prime editing, the Cas9 nickase cuts the non-complementary strand of DNA upstream of the PAM site, thereby providing a 3′ flap that is extended with the extension including a modification. Further details on Prime editing can be found in Anzalone et al. (2019) Nature 576, 149-157 and/or U.S. Patent Application Publication No. 2021/0147862, the contents of each of which are incorporated herein by reference in their entirety.

In some embodiments, an editing system of the present invention incorporates the Redraw editing system. Further details on the Redraw editing system can be found in U.S. Patent Application Publication No. 2021/0130835 and/or in U.S. Patent Application Publication No. 2022/0145334, the contents of each of which are incorporated herein by reference in their entirety.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target nucleic acid (e.g., a target DNA and/or a protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas12f, Cas12i, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, Csx10, Csx16, CsaX, Csx3, Csxl, Csx15, Csfl, Csf2, Csf3, Csf4 (dinG), and/or Csf5, or a fragment thereof), wherein the repeat sequence may be linked to the 5′ end and/or 3′ end of the spacer sequence. In some embodiments, the guide nucleic acid comprises DNA. In some embodiments, the guide nucleic acid comprises RNA (e.g., is a guide RNA). The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35 (Web Server issue): W52-7). In some embodiments, a repeat sequence or portion thereof is linked at its 3′ end to 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein; e.g., about). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild-type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild-type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer). The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length.

In some embodiments, 5′ region of a spacer sequence of a guide nucleic acid may be fully complementary to a target nucleic acid, while 3′ region of the spacer may be substantially complementary to the target nucleic acid (such as for a spacer in a Type V CRISPR-Cas system), or 3′ region of a spacer sequence of a guide nucleic acid may be fully complementary to a target nucleic acid, while 5′ region of the spacer may be substantially complementary to the target nucleic acid (such as for a spacer in a Type II CRISPR-Cas system), and therefore, the overall complementarity of the spacer sequence to the target nucleic acid may be less than 100%. Thus, for example, in a guide nucleic acid for a Type V CRISPR-Cas system, the first 1, 2, 3, 4, 5, 6, 7, 8, 9, 10 nucleotides in 5′ region (i.e., seed region) of, for example, a 20 nucleotide spacer sequence may be 100% complementary to the target nucleic acid, while the remaining nucleotides in 3′ region of the spacer sequence are substantially complementary (e.g., at least about 70% complementary) to the target nucleic acid. In some embodiments, the first 1 to 8 nucleotides (e.g., the first 1, 2, 3, 4, 5, 6, 7, 8, nucleotides, and any range therein) of 5′ end of the spacer sequence may be 100% complementary to the target nucleic acid, while the remaining nucleotides in 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target nucleic acid.

As a further example, in a guide nucleic acid for a Type II CRISPR-Cas system, the first 1, 2, 3, 4, 5, 6, 7, 8, 9, 10 nucleotides in 3′ region (i.e., seed region) of, for example, a 20 nucleotide spacer sequence may be 100% complementary to the target nucleic acid, while the remaining nucleotides in 5′ region of the spacer sequence are substantially complementary (e.g., at least about 70% complementary) to the target nucleic acid. In some embodiments, the first 1 to 10 nucleotides (e.g., the first 1, 2, 3, 4, 5, 6, 7, 8, 9, 10 nucleotides, and any range therein) of 3′ end of the spacer sequence may be 100% complementary to the target nucleic acid, while the remaining nucleotides in 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target nucleic acid. A recruiting guide RNA further comprises one or more recruiting motifs as described herein, which may be linked to 5′ end of the guide or 3′ end or it may be inserted into the recruiting guide nucleic acid (e.g., within the hairpin loop).

A “recruiting motif” as used herein refers to one half of a binding pair that may be used to recruit a compound to which the recruiting motif is bound to another compound that includes the other half of the binding pair (i.e., a “corresponding motif”). The recruiting motif and corresponding motif may bind covalently and/or noncovalently. In some embodiments, a recruiting motif is an RNA recruiting motif (e.g., an RNA recruiting motif that is capable of binding and/or configured to bind to an affinity polypeptide), an affinity polypeptide (e.g., an affinity polypeptide that is capable of binding and/or configured to bind an RNA recruiting motif and/or a peptide tag), or a peptide tag (e.g., a peptide tag that is capable of binding and/or configured to bind an affinity polypeptide). For example, when a recruiting motif is an RNA recruiting motif, the corresponding motif for the RNA recruiting motif may be an affinity polypeptide that binds the RNA recruiting motif. A further example is that when a recruiting motif is a peptide tag, the corresponding motif for the peptide tag may be an affinity polypeptide that binds the peptide tag. Thus, a compound comprising a recruiting motif (e.g., an affinity polypeptide) may be recruited to another compound (e.g., a guide nucleic acid) comprising a corresponding motif for the recruiting motif (e.g., an RNA recruiting motif). Exemplary peptide tags (e.g., epitope) include, but are not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope.

In some embodiments, a seed region of a spacer may be about 8 to about 10 nucleotides in length, about 5 to about 6 nucleotides in length, or about 6 nucleotides in length.

A “target nucleic acid”, “target DNA,” “target nucleotide sequence,” “target region,” and “target region in the genome” are used interchangeably herein and refer to a region of an organism's (e.g., a plant's) genome that comprises a sequence that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid as defined herein. A target nucleic acid is targeted by an editing system (or a component thereof) as described herein. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome or mammalian (e.g., human) genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” or “protospacer” as used herein refer to a sequence that is fully or substantially complementary to (and can hybridize to) a spacer sequence of a guide nucleic acid. In some embodiments, the protospacer is all or a portion of a target nucleic acid as defined herein that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at 3′ end of the target strand (see below, as an example).

```
    5′-NNNNNNNNNNNNNNNNNNN-3′ RNA Spacer
            ||||||||||||||||||
3′AAANNNNNNNNNNNNNNNNNNN-5′ Target strand
    ||||
5′TTTNNNNNNNNNNNNNNNNNNN-3′ Non-target strand
```

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, a guide nucleic acid further comprises a reverse transcriptase template and may be referred to as an extended guide nucleic acid. An “extended guide nucleic acid” as used herein is a guide nucleic acid as described herein that further comprises a reverse transcriptase template (RTT) and/or a primer binding site (PBS). In some embodiments, an extended guide nucleic acid is an engineered prime editing guide RNA (pegRNA). An extended guide nucleic acid may be a targeted allele guide RNA (tagRNA) or a stabilized targeted allele guide RNA (stagRNA). A “tagRNA” as used herein refers to an extended guide nucleic acid that comprises a PBS and a RTT and has target strand complementarity. A “stagRNA” as used herein refers to a tagRNA that comprises a stabilization motif. A stabilization motif may be present at 3′ and/or 5′ end of a tagRNA. In some embodiments, a stabilization motif is present at 3′ end of a tagRNA. Exemplary stabilization motifs include, but are not limited to, recruiting motifs, RNA hairpins, pseudoknot sequences, and/or PP7 motifs (e.g., a PP7 RNA hairpin sequence). In some embodiments, a stagRNA is a tagRNA that comprises a PP7 RNA hairpin sequence. In some embodiments, a CRISPR-Cas effector protein (e.g., a Type II or Type V CRISPR-Cas effector protein), a reverse transcriptase, and an extended guide nucleic acid can form a complex or are comprised in a complex.

In some embodiments, an extended guide nucleic acid comprises an extended portion that includes a primer binding site and a reverse transcriptase template, wherein the reverse transcriptase template comprises the modification (e.g., edit) to be incorporated into a target nucleic acid. In some embodiments, an extended guide nucleic acid comprises, at its 3′ end, a primer binding site and a modification (e.g., an edit) to be incorporated into the target nucleic acid (e.g., a reverse transcriptase template). In some embodiments, an extended guide nucleic acid comprises: (1) a sequence that interacts (e.g., recruits and/or binds) with a CRISPR-Cas effector protein (e.g., a CRISPR-Cas nuclease), (2) a spacer having substantial complementary to a first site on a target nucleic acid (e.g., a CRISPR RNA (crRNA) (a first crRNA) and/or tracrRNA+crRNA (sgRNA)), and (3) a nucleic acid encoded repair template (e.g., an RNA encoded repair template) comprising a primer binding site and an RNA template (e.g., that encodes the modification to be incorporated into the target nucleic acid). In some embodiments, an extended guide nucleic acid (e.g., an extended guide RNA) may comprise, 5′-3′, a spacer sequence, a repeat sequence, and an extended portion, the extended portion comprising, 5′ to 3′, a reverse transcriptase template and a primer binding site. In some embodiments, an extended guide nucleic acid may comprise, 5′-3′, a spacer sequence, a repeat sequence and an extended portion, the extended portion comprising, 5′ to 3′, a primer binding site and a reverse transcriptase template. In some embodiments, an extended guide nucleic acid may comprise, 5′-3′, an extended portion, a spacer sequence, and a repeat sequence, wherein the extended portion comprises, 5′ to 3′, a reverse transcriptase template and a primer binding site. In some embodiments, an extended guide nucleic acid may comprise, 5′-3′, an extended portion, a spacer sequence, and a repeat sequence, wherein the extended portion comprises, 5′ to 3′, a primer binding site and a reverse transcriptase template.

According to some embodiments, an extended guide nucleic acid (e.g., a pegRNA) may have a structure and/or be designed as described in Anzalone et al., Nature, 2019 December; 576 (7785): 149-157. In some embodiments, an extended guide nucleic acid comprises a primer binding site (PBS) optionally having a sequence of 1, 2, 3, 4, 5, 6, 7, 8, 9, 10, 11, 12, 13, 14, or 15 nucleotides and a reverse transcriptase template (RT template) sequence optionally having a sequence of 65 nucleotides or more. In some embodiments, a PBS of an extended guide nucleic acid has a sequence of less than 15 nucleotides and has a sequence of 1, 2, 3, 4, 5, 6, 7, 8, 9, 10, 11, 12, 13, or 14 nucleotides (e.g., a sequence of 5 or 6 nucleotides in length). The RT template sequence may be after the PBS sequence in 5′ to 3′ direction. In some embodiments, the RT template sequence of the extended guide nucleic acid has a length of greater than 65 nucleotides and may comprise about 50 or more nucleotides of heterology relative to the target site (e.g., target nucleic acid), followed by about 15 or more nucleotides of homology relative to the target site. In some embodiments, the RT template sequence of the extended guide nucleic acid is after the PBS sequence and the RT template sequence has a length of greater than 65 nucleotides with the sequence including more than 50 nucleotides of heterology relative to the target site, followed by more than 15 nucleotides of homology relative to the target site. Accordingly, in some embodiments, when the extended guide nucleic acid is reverse transcribed, the resulting newly transcribed sequence may hybridize and/or is configured to hybridize with the unnicked strand of the target site, which may thereby create a heteroduplex DNA with a large insertion into the newly synthesized strand. Upon repair of this mismatched DNA, the resultant repaired DNA may contain a large insertion (e.g., greater than 50 nucleotides) of DNA sequence. In some embodiments, the method may provide a large deletion (e.g., greater than 50 nucleotides) of DNA sequence. In some embodiments, the PBS and the 15 or more nucleotides of homology to the target site may comprise homology arms, which may serve to insert the heterology into the target site optionally using homology directed repair. The inserted DNA may correspond to any functional sequence of DNA such as, but not limited to: a functional transgene; a fragment of DNA that is inserted into a gene in a way that, when the gene is transcribed, would produce a hairpin RNA that is sufficient to silence homologous genes through RNAi; and/or one or more functional site-specific recombination sites, e.g. lox, frt, which could then be used in subsequent Cre or Flp mediated site-specific recombination processes. In some embodiments, an extended guide nucleic acid may be too large to produce using a PolIII promoter in vivo. In some embodiments, an extended guide nucleic acid may be operatively associated with and/or produced using a PollI promoter. In some embodiments, a DNA binding polypeptide (e.g., a DNA binding domain) and/or DNA endonuclease may have a structure and/or be designed as described in Anzalone et al., Nature, 2019 December; 576 (7785): 149-157. In some embodiments, a DNA binding domain and/or DNA endonuclease is a CRISPR Cas polypeptide such as a Cas9 nickase, a nicking variant of another CRISPR Cas polypeptide, or Cas12a.

In some embodiments, two extended guide nucleic acids (e.g., pegRNAs) may be used (e.g., an editing system may comprise two extended guide nucleic acids). One or both of the two extended guide nucleic acids may have a structure and/or be designed as described in Anzalone et al., Nature, 2019 December; 576 (7785): 149-157. The two extended guide nucleic acids may comprise a primer binding site (PBS) optionally having a sequence of 1, 2, 3, 4, 5, 6, 7, 8, 9, 10, 11, 12, 13, 14, or 15 nucleotides and a reverse transcriptase template (RT template) sequence optionally having a sequence of 50 nucleotides or more. The RT template sequences of the two extended guide nucleic acids may be complementary to each other and as such the polynucleotides that are respectively reverse transcribed from each the RT templates will be complementary to each other and will be able to hybridize with each other. This may allow for the intermediates that are produced by this system and/or method to join together two sections of DNA that are otherwise separated by more than 50 nucleotides, e.g., within a chromosome, or that are positioned on two separate pieces of DNA, e.g., on two different chromosomes. After repair of the intermediates, the resultant products may produce, depending on the design of the RT template, large deletions, large inversions, or inter-chromosomal recombinations. Since all of these products are produced by homology directed repair, the products may be predictably precise and/or reproducible. In some embodiments, a DNA binding polypeptide (e.g., a DNA binding domain) and/or DNA endonuclease may have a structure and/or be designed as described in Anzalone et al., Nature, 2019 December; 576 (7785): 149-157. In some embodiment, a DNA binding polypeptide and/or DNA endonuclease is a CRISPR Cas polypeptide such as a Cas9 nickase, a similar nicking variant of another CRISPR Cas polypeptide, or Cas12a. In some embodiments, a DNA binding polypeptide and/or DNA endonuclease is a Cas9 nuclease, a similar nuclease from another CRISPR Cas polypeptide, or Cas12a. Using a nuclease (rather than a nickase) may facilitate the intra- or interchromosomal recombination processes through single-strand annealing of the more than 50 nucleotide 3′ overhangs that would be produced at each of the two target sites corresponding to the two pegRNA target nucleic acids. In some embodiments, an editing system comprises one extended guide nucleic acid and a guide nucleic acid that is devoid of a reverse transcriptase template and/or primer binding site.

An extended guide nucleic acid may comprise a CRISPR nucleic acid (e.g., CRISPR RNA, CRISPR DNA, crRNA, crDNA) and/or a CRISPR nucleic acid and a tracr nucleic acid; and (b) an extended portion comprising a primer binding site and a reverse transcriptase template (RT template), wherein the RT template encodes a modification to be incorporated into the target nucleic acid. The CRISPR nucleic acid may be a Type II or Type V CRISPR nucleic acid and/or the tracr nucleic acid may be any tracr corresponding to the appropriate Type II or Type V CRISPR nucleic acid. In some embodiments, an extended guide nucleic acid comprises: (i) a Type V CRISPR nucleic acid or a Type II CRISPR nucleic acid (e.g., a Type II or Type V CRISPR RNA, Type II or Type V CRISPR DNA, Type II or Type V crRNA, or Type II or Type V crDNA) and/or a CRISPR nucleic acid and a tracr nucleic acid (e.g., a Type II or Type V tracrRNA, Type II or Type V tracrDNA); and (ii) an extended portion comprising a primer binding site and a reverse transcriptase template (RT template), wherein the Type V CRISPR nucleic acid or Type II CRISPR nucleic acid comprises a spacer that binds to a first strand (e.g., the target strand) of a target nucleic acid (e.g., the spacer is complementary to a portion of consecutive nucleotides in the first strand of the target nucleic acid) and the primer binding site binds to the first strand (e.g., target strand). In some embodiments, the extended portion can be fused to either 5′ end or 3′ end of the CRISPR nucleic acid (e.g., from 5′ to 3′: repeat-spacer-extended portion or extended portion-repeat-spacer) and/or to the 5′ or 3′ end of the tracr nucleic acid. In some embodiments, the extended portion of an extended guide nucleic acid comprises, 5′ to 3′, an RT template (RTT) and a primer binding site (PBS) (e.g., 5′-crRNA-spacer-RTT (edit encoded)-PBS-3′) or comprises 5′ to 3′ a PBS and RTT, depending on the location of the extended portion relative to the CRISPR nucleic acid of the extended guide nucleic acid (e.g., 5′-crRNA-spacer-PBS-RTT (edit encoded)-3′). For example, in some embodiments, an extended portion of the extended guide nucleic acid may comprise, 5′ to 3′, an RT template and a primer binding site (when the extended guide is linked to the 3′ end of the CRISPR nucleic acid). In some embodiments, an extended portion of the extended guide may comprise, 5′ to 3′, a primer binding site and an RT template (when the extended guide is linked to the 5′ end of the CRISPR nucleic acid).

In some embodiments, a target nucleic acid is double stranded and comprises a first strand and a second strand and a primer binding site of an extended guide nucleic acid binds to the second strand (e.g., the non-target, top strand) of the target nucleic acid. In some embodiments, a target nucleic acid is double stranded and comprises a first strand and a second strand and a primer binding site of an extended guide nucleic acid binds to the first strand (e.g., binds to the target strand, optionally the same strand to which a CRISPR-Cas effector protein is recruited, bottom strand) of the target nucleic acid. In some embodiments, a target nucleic acid is double stranded and comprises a first strand and a second strand and the primer binding site of an extended guide nucleic acid binds to the second strand (e.g., the non-target strand, optionally the opposite strand from that to which the CRISPR-Cas effector protein is recruited) of the target nucleic acid. In some embodiments, a reverse transcriptase (RT) may add to the target strand of a target nucleic acid (e.g., the strand to which the spacer of the CRISPR nucleic acid of the extended guide nucleic acid is complementary and to which the CRISPR-Cas effector protein is recruited). In some embodiments, the reverse transcriptase (RT) adds to the non-target strand of a target nucleic acid (e.g., the strand that is complementary to the strand to which the spacer of the CRISPR nucleic acid is complementary and to which the CRISPR-Cas effector protein is recruited). Example methods and editing systems are described in International Patent Publication No. WO 2021/092130, International Patent Publication No. WO 2022/098993, and U.S. Patent Application Publication Nos. 2021/0147862, 2021/0130835, 2021/0147862, and 2022/0145334, each of which are incorporated herein by reference in their entirety.

The RT template of an extended guide nucleic acid may encode one or more modification(s) (e.g., edit(s)) to be incorporated into a target nucleic acid. The one or more modification(s) may be located in any position within an RT template (e.g., where the position location may be relative to the position of a protospacer adjacent motif (PAM) of the target nucleic acid). In some embodiments, an RT template has a modification at one or more positions from −1 to 23 (e.g., −1, 1, 2, 3, 4, 5, 6, 7, 8, 9, 10, 11, 12, 13, 14, 15, 16, 17, 18, 19, 20, 21, 22, or 23) relative to the position of a protospacer adjacent motif (PAM) (e.g., TTTG) in a target nucleic acid. In some embodiments, an RT template may comprise a modification located at nucleotide position −1, 1, 2, 3, 4, 5, 6, 7, 8, 9, 10, 11, 12, 13, 14, 15, 16, 17, 18, 19, 20, 21, 22 or 23. In some embodiments, an RT template may comprise a modification located at nucleotide position 4 to nucleotide position 17 (e.g., position 4, 5, 6, 7, 8, 9, 10, 11, 12, 13, 14, 15, 16, or 17) of the RT template relative to the position of a PAM of a target nucleic acid. In some embodiments, an RT template may comprise a modification located at nucleotide position 10 to nucleotide position 17 (e.g., position 10, 11, 12, 13, 14, 15, 16, or 17) of the RT template relative to the position of a PAM of a target nucleic acid. In some embodiments, an RT template may comprise a modification located at nucleotide position 12 to nucleotide position 15 (e.g., position 12, 13, 14, or 15) of the RT template relative to the position of a PAM of a target nucleic acid.

In some embodiments, an extended portion of an extended guide nucleic acid may comprise, 5′ to 3′, an RT template and a primer binding site (e.g., when the extended portion is linked to the 3′ end of a CRISPR nucleic acid). In some embodiments, an extended portion of an extended guide nucleic acid may comprise, 5′ to 3′, a primer binding site and an RT template (RTT) (e.g., when the extended portion is linked to the 5′ end of the CRISPR nucleic acid). In some embodiments, an RT template may have a length of about 1 nucleotide to about 100 nucleotides (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides, and any range or value therein), e.g., about 1 nucleotide to about 10 nucleotides, about 1 nucleotide to about 15 nucleotides, about 1 nucleotide to about 20 nucleotides, about 1 nucleotide to about 25 nucleotides, about 1 nucleotide to about 30 nucleotides, about 1 nucleotide to about 35, 36, 37, 38, 39 or 40 nucleotides, about 1 nucleotide to about 50 nucleotides, about 5 nucleotides to about 15 nucleotides, about 5 nucleotides to about 20 nucleotides, about 5 nucleotides to about 25 nucleotides, about 5 nucleotides to about 30 nucleotides, about 5 nucleotides to about 35, 36, 37, 38, 39 or 40 nucleotides, about 5 nucleotides to about 50 nucleotides, about 8 nucleotides to about 15 nucleotides, about 8 nucleotide to about 20 nucleotides, about 8 nucleotide to about 25 nucleotides, about 8 nucleotide to about 30 nucleotides, about 8 nucleotide to about 35, 36, 37, 38, 39 or 40 nucleotides, about 8 nucleotide to about 50 nucleotides in length, about 8 nucleotides to about 100 nucleotides, about 10 nucleotide to about 15 nucleotides, about 10 nucleotide to about 20 nucleotides, about 10 nucleotide to about 25 nucleotides, about 10 nucleotide to about 30 nucleotides, about 10 nucleotide to about 36 nucleotides, about 10 nucleotide to about 40 nucleotides, about 10 nucleotide to about 50 nucleotides, about 10 nucleotides to about 100 nucleotides in length and any range or value therein. In some embodiments, the length of an RT template may be at least 8 nucleotides, optionally about 8 nucleotides to about 100 nucleotides. In some embodiments, the length of an RT template is 36, 37, 38, 39 or 40 nucleotides or less (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length, or any value or range therein (e.g., about 1, 2, 3, 4, 5, 6, 7, 8, 9, 10, 11, 12, 13, 14, or 15 nucleotides in length to about 16, 17, 18, 19, 20, 21, 22, 23, 24, 25, 26, 27, 28, 29, 30, 31, 32, 33, 34, 35, 36, 37, 38, 39, or 40 nucleotides in length). In some embodiments, the length of an RT template may be at least 30 nucleotides, optionally about 30, 31, 32, 33, 34, 35, 36, 37, 38, 39, or 40 nucleotides in length to about to about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length, or any range or value therein. In some embodiments, the length of an RT template may be about 36, 40, 44, 47, 50, 52, 55, 63, 72 or 74 nucleotides. Within the length of the RTT one or more modification(s) may be present. The one or more modification(s) may be located anywhere within the RTT, wherein the position of the modification may be described relative to the position of a protospacer adjacent motif (PAM) of a target nucleic acid. In some embodiments, an RT template may comprise a modification located at nucleotide position −1, 1, 2, 3, 4, 5, 6, 7, 8, 9, 10, 11, 12, 13, 14, 15, 16, 17, 18, 19, 20, 21, 22 or 23. In some embodiments, an RT template may comprise a modification located at nucleotide position 4 to nucleotide position 17 (e.g., position 4, 5, 6, 7, 8, 9, 10, 11, 12, 13, 14, 15, 16, or 17) of the RT template relative to the position of a protospacer adjacent motif (PAM) of a target nucleic acid. In some embodiments, an RT template may comprise a modification located at nucleotide position 10 to nucleotide position 17 (e.g., position 10, 11, 12, 13, 14, 15, 16, or 17) of the RT template relative to the position of a protospacer adjacent motif (PAM) of a target nucleic acid. In some embodiments, an RT template may comprise a modification located at nucleotide position 12 to nucleotide position 15 (e.g., position 12, 13, 14, or 15) of the RT template relative to the position of a protospacer adjacent motif (PAM) of a target nucleic acid.

As used herein, a “primer binding site” (PBS) of an extended portion of an extended guide nucleic acid (e.g., a tagRNA) refers to a sequence of consecutive nucleotides that can bind to a region or “primer” on a target nucleic acid, e.g., is complementary to the target nucleic acid primer. As an example, a CRISPR Cas effector protein (e.g., a Type II or Type V, e.g., Cas 9 or Casl2a) may nick/cut the DNA and 3′ end of the cut DNA acts as a primer for the PBS portion of the extended guide nucleic acid. The PBS may be complementary to the 3′ end of a strand of the target nucleic acid and may bind and/or may be configured to bind to either the target strand or non-target strand. A primer binding site can be fully complementary to the primer or it may be substantially complementary (e.g., at least 70% complementary (e.g., 70% or about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9% or more)) to the primer of a target nucleic acid. In some embodiments, the length of a primer binding site of an extended portion may be about 1 nucleotide to about 100 nucleotides in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides, or any value or range therein), or about 4 nucleotide to about 85 nucleotides, about 10 nucleotide to about 80 nucleotides, about 20 nucleotide to about 80 nucleotides, about 25 nucleotides to about 80 nucleotides about 30 nucleotide to about 80 nucleotides, about 40 nucleotide to about 80 nucleotides, about 45 nucleotide to about 80 nucleotides, about 45 nucleotide to about 75 nucleotides, or about 45 nucleotide to about 60 nucleotides, or any range or value therein. In some embodiments, the length of a PBS may be at least 30 nucleotides, optionally about 30, 31, 32, 33, 34, 35, 36, 37, 38, 39, or 40 nucleotides to about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides in length, or any range or value therein. In some embodiments, the length of a PBS may be about 8, 16, 24, 32, 40, 48, 56, 64, 72, or 80 nucleotides.

In some embodiments, an RTT may have a length of about 35 nucleotides to about 75 nucleotides and a PBS may have a length of about 30 nucleotides to about 80 nucleotides, optionally wherein the PBS may comprise a length of about 8, 16, 24, 32, 40, 48, 56, 64, 72, or 80 nucleotides and the RTT may comprise a length of about 36, 40, 44, 47, 50, 52, 55, 63, 72 or 74 nucleotides, or any combination thereof of the RTT length and/or PBS length.

In some embodiments, an extended portion of an extended guide nucleic acid may be fused to either 5′ end or 3′ end of a Type II or a Type V CRISPR nucleic acid (e.g., 5′ to 3′: repeat-spacer-extended portion, or extended portion-repeat-spacer) and/or to 5′ or 3′ end of the tracr nucleic acid. In some embodiments, when an extended portion is located 5′ of the crRNA, a Type V CRISPR-Cas effector protein is modified to reduce (or eliminate) self-processing RNAse activity.

In some embodiments, the extended portion of an extended guide nucleic acid may be linked to the Type II or Type V CRISPR nucleic acid and/or the Type II or Type V tracrRNA via a linker. In some embodiments, a linker have a length of about 1 to about 100 nucleotides or more (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length, and any range therein (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, about 40 to about 100, about 50 to about 100, or about 2, 3, 4, 5, 6, 7, 8, 9, 10, 11, 12, 13, 14, 15, 16, 17, 18, 19, 20, 21, 22, 23, 24, 25 nucleotides to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length (e.g., about 105, 110, 115, 120, 130, 140 150 or more nucleotides in length).

A guide nucleic acid and/or an extended guide nucleic acid may comprise one or more recruiting motifs as described herein, which may be linked to the 5′ end and/or 3′ end of the guide nucleic acid and/or it may be inserted into the guide nucleic acid (e.g., within a hairpin loop of the guide nucleic acid). In some embodiments, an extended guide nucleic acid may be linked to an RNA recruiting motif. An extended guide nucleic acid and/or guide nucleic acid may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif may be located on 3′ end of the extended portion of an extended guide nucleic acid (e.g., 5′-3′, repeat-spacer-extended portion (RT template-primer binding site)-RNA recruiting motif). In some embodiments, an RNA recruiting motif may be embedded in the extended portion of an extended guide nucleic acid.

In some embodiments, an editing system comprises an extended guide nucleic acid that is linked to an RNA recruiting motif and a reverse transcriptase that is a reverse transcriptase fusion protein, wherein the reverse transcriptase fusion protein comprises a reverse transcriptase polypeptide fused to an affinity polypeptide that binds to the RNA recruiting motif, wherein the extended guide nucleic acid binds to a target nucleic acid and the RNA recruiting motif binds to the affinity polypeptide, thereby recruiting the reverse transcriptase fusion protein to the extended guide nucleic acid and contacting the target nucleic acid with the reverse transcriptase. In some embodiments, two or more reverse transcriptase fusion proteins may be recruited to an extended guide nucleic acid, thereby contacting the target nucleic acid with two or more reverse transcriptase fusion proteins.

The terms “transgene” or “transgenic” as used herein refer to at least one nucleic acid sequence that is taken from the genome of one organism or produced synthetically, and which is then introduced into a host cell (e.g., a plant cell) or organism or tissue of interest and which is subsequently integrated into the host's genome by means of “stable” transformation or transfection approaches. In contrast, the term “transient” transformation or transfection or introduction refers to a way of introducing molecular tools including at least one nucleic acid (DNA, RNA, single-stranded or double-stranded or a mixture thereof) and/or at least one amino acid sequence, optionally comprising suitable chemical or biological agents, to achieve a transfer into at least one compartment of interest of a cell, including, but not restricted to, the cytoplasm, an organelle, including the nucleus, a mitochondrion, a vacuole, a chloroplast, or into a membrane, resulting in transcription and/or translation and/or association and/or activity of the at least one molecule introduced without achieving a stable integration or incorporation into the genome and thus without inheritance of the respective at least one molecule introduced into the genome of a cell. The term “transgene-free” refers to a condition in which a transgene is not present or found in the genome of a host cell or tissue or organism of interest.

As used herein, a “CRISPR-Cas effector protein” is a protein or polypeptide that cleaves, cuts, or nicks a nucleic acid; binds a nucleic acid (e.g., a target nucleic acid and/or a guide nucleic acid); and/or that identifies, recognizes, or binds a guide nucleic acid as defined herein. In some embodiments, a CRISPR-Cas effector protein may be an enzyme (e.g., a nuclease, endonuclease, nickase, etc.) and/or may function as an enzyme. In some embodiments, a CRISPR-Cas effector protein refers to a CRISPR-Cas nuclease. In some embodiments, a CRISPR-Cas effector protein comprises nuclease activity and/or nickase activity, comprises a nuclease domain whose nuclease activity and/or nickase activity has been reduced or eliminated, comprises single stranded DNA cleavage activity (ss DNAse activity) or which has ss DNAse activity that has been reduced or eliminated, and/or comprises self-processing RNAse activity or which has self-processing RNAse activity that has been reduced or eliminated. A CRISPR-Cas effector protein may bind to a target nucleic acid. A CRISPR-Cas effector protein may be a Type I, II, III, IV, V, or VI CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be a Type II CRISPR-Cas effector protein, for example, a Cas9 effector protein. In some embodiments, a CRISPR-Cas effector protein may be Type V CRISPR-Cas effector protein, for example, a Cas12 effector protein.

Exemplary CRISPR-Cas effector proteins include, but are not limited to, a Cas9, C2c1, C2c3, Cas12a (also referred to as Cpf1), Cas12b, Cas12c, Cas12d, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csy1, Csy2, Csy3, Csel, Cse2, Csel, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, Csx10, Csx16, CsaX, Csx3, Csxl, Csx15, Csfl, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site and/or nuclease domain (e.g., a RuvC, HNH, e.g., a RuvC site of a Cas12a nuclease domain; e.g., a RuvC site and/or HNH site of a Cas9 nuclease domain). A CRISPR-Cas effector protein having a mutation in its nuclease active site and/or nuclease domain, and therefore, no longer comprising nuclease activity, is commonly referred to as “inactive” or “dead,” e.g., dCas9. In some embodiments, a CRISPR-Cas effector protein having a mutation in its nuclease active site and/or nuclease domain may have impaired activity or reduced activity (e.g., nickase activity) as compared to the same CRISPR-Cas effector protein without the mutation.

A CRISPR Cas9 effector protein or Cas9 useful with this invention may be any known or later identified Cas9 nuclease. In some embodiments, a Cas9 of the present invention may be a protein from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp.

In some embodiments, the CRISPR-Cas effector protein may be a Cas9 derived from Streptococcus pyogenes and/or may recognize the PAM sequence motif NGG, NAG, NGA (Mali et al, Science 2013; 339 (6121): 823-826). In some embodiments, the CRISPR-Cas effector protein may be a Cas9 derived from Streptococcus thermophiles and/or may recognize the PAM sequence motif NGGNG and/or NNAGAAW (W=A or T) (See, e.g., Horvath et al, Science, 2010; 327 (5962): 167-170, and Deveau et al, J Bacteriol 2008; 190 (4): 1390-1400). In some embodiments, the CRISPR-Cas effector protein may be a Cas9 derived from Streptococcus mutans and/or may recognize the PAM sequence motif NGG and/or NAAR (R=A or G) (See, e.g., Deveau et al, J BACTERIOL 2008; 190 (4): 1390-1400). In some embodiments, the CRISPR-Cas effector protein may be a Cas9 derived from Streptococcus aureus and/or may recognize the PAM sequence motif NNGRR (R=A or G). In some embodiments, the CRISPR-Cas effector protein may be a Cas9 derived from S. aureus and/or may recognize the PAM sequence motif N GRRT (R=A or G). In some embodiments, the CRISPR-Cas effector protein may be a Cas9 derived from S. aureus and/or may recognize the PAM sequence motif N GRRV (R=A or G). In some embodiments, the CRISPR-Cas effector protein may be a Cas9 that is derived from Neisseria meningitidis and/or may recognize the PAM sequence motif N GATT or N GCTT (R=A or G, V=A, G or C) (See, e.g., Hou et ah, PNAS 2013, 1-6). In the aforementioned embodiments in this paragraph, N in the PAM sequence motif can be any nucleotide residue, e.g., any of A, G, C or T. In some embodiments, the CRISPR-Cas effector protein may be a Cas13a derived from Leptotrichia shahii and/or may recognize a protospacer flanking sequence (PFS) (or RNA PAM (rPAM)) sequence motif of a single 3′ A, U, or C, which may be located within the target nucleic acid.

A Type V CRISPR-Cas effector protein useful with embodiments of the invention may be any Type V CRISPR-Cas nuclease. Exemplary Type V CRISPR-Cas effector proteins include, but are not limited, to Cas12a (Cpf1), Cas12b, Cas12c (C2c3), Cas12d (CasY), Cas12e (CasX), Cas12g, Cas12h, Cas12i, C2c1, C2c4, C2c5, C2c8, C2c9, C2c10, Cas14a, Cas14b, and/or Cas14c nuclease. In some embodiments, a Type V CRISPR-Cas effector protein may be a Cas12a. In some embodiments, a Type V CRISPR-Cas effector protein may be a nickase, optionally, a Cas12a nickase.

In some embodiments, the CRISPR-Cas effector protein may be a Type V Clustered Regularly Interspaced Short Palindromic Repeats (CRISPR)-Cas nuclease. Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein useful with this invention may be any known or later identified Cas12a (previously known as Cpf1) (see, e.g., U.S. Pat. No. 9,790,490, which is incorporated by reference for its disclosures of Cpf1 (Cas12a) sequences). The term “Cas12a” refers to an RNA-guided protein that can have nuclease activity, the protein comprising a guide nucleic acid binding domain and an active, inactive, or partially active DNA cleavage domain, thereby the RNA-guided nuclease activity of the Cas12a may be active, inactive or partially active, respectively. In some embodiments, a Cas12a useful with the invention may comprise a mutation in the nuclease active site (e.g., a RuvC site of the Cas12a domain). A Cas12a having a mutation in its nuclease domain and/or nuclease active site, and therefore, no longer comprising nuclease activity, is commonly referred to as deadCas 12a (e.g., dCas12a). In some embodiments, a Cas12a having a mutation in its nuclease domain and/or nuclease active site may have impaired activity, e.g., may have reduced nickase activity.

In some embodiments, a CRISPR-Cas effector protein may be optimized for expression in an organism, for example, in an animal (e.g., a mammal such as a human), a plant, a fungus, an archaeon, or a bacterium. In some embodiments, a CRISPR-Cas effector protein (e.g., Cas12a polypeptide/domain or a Cas9 polypeptide/domain) may be optimized for expression in a plant.

In some embodiments, a CRISPR-Cas effector protein comprising a Cas9 domain (or a nucleic acid construct encoding the same) may be used in combination with a Cas9 guide nucleic acid to modify a target nucleic acid, and may be in or may form a complex.

Likewise, a CRISPR-Cas effector protein may comprise a Cas12a domain (or other selected CRISPR-Cas nuclease, e.g., C2c1, C2c3, Cas12b, Cas12c, Cas12d, Cas12f, Cas12i, Cas12e, Cas13a, Cas13b, Cas13c, Cas13d, Casl, CaslB, Cas2, Cas3, Cas3′, Cas3″, Cas4, Cas5, Cas6, Cas7, Cas8, Cas9 (also known as Csnl and Csx12), Cas10, Csy1, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, Csx10, Csx16, CsaX, Csx3, Csxl, Csx15, Csfl, Csf2, Csf3, Csf4 (dinG), and/or Csf5), which may be used in combination with a Cas12a guide nucleic acid (or the guide nucleic acid for the other selected CRISPR-Cas nuclease) to modify a target nucleic acid, thereby editing the target nucleic acid.

As used herein, “modifying” or “modification” in reference to a target nucleic acid includes editing (e.g., mutating), covalent modification, exchanging/substituting nucleic acids/nucleotide bases, deleting, cleaving, and/or nicking of a target nucleic acid to thereby provide a modified nucleic acid and/or altering transcriptional control of a target nucleic acid to thereby provide a modified nucleic acid. In some embodiments, a modification may include an insertion and/or deletion of any size and/or a single base change (SNP) of any type. In some embodiments, a modification comprises a SNP. In some embodiments, a modification comprises exchanging and/or substituting one or more (e.g., 1, 2, 3, 4, 5, or more) nucleotides. In some embodiments, an insertion or deletion may be about 1 base to about 30,000 bases or more in length (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, 30,000 bases in length or more, or any value or range therein). Thus, in some embodiments, an insertion or deletion may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 to about 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 bases in length, or any range or value therein; about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300 bases to about 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 bases or more in length, or any value or range therein; about 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000 bases to about 2500, 3000, 3500, 4000, 4500, 5000, 5500, 6000, 6500, 7000, 7500, 8000, 8500, 9000, 9500, or 10,000 bases or more in length, or any value or range therein; or about 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, or 700 bases to about 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2500, 3000, 3500, 4000, 4500, or 5000 bases or more in length, or any value or range therein. In some embodiments, an insertion or deletion may be about 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, or 10,000 bases to about 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, or 30,000 bases or more in length, or any value or range therein.

“Introducing,” “introduce,” “introduced” (and grammatical variations and derivatives thereof) in the context of a polynucleotide or editing system means presenting a nucleotide sequence of interest (e.g., polynucleotide, a nucleic acid construct, and/or a guide nucleic acid) and/or editing system (e.g., a polynucleotide, polypeptide, and/or ribonucleoprotein) to a host organism or cell of said organism (e.g., host cell; e.g., a plant cell) in such a manner that the nucleotide sequence and/or editing system gains access to the interior of a cell. Thus, for example, a polynucleotide from an Agrobacterium strain, such as A. tumefaciens or A. rhizogenes, (that may encode or comprise at least a portion of an editing system) may be introduced into a cell of an organism, thereby transforming the cell with the polynucleotide. In some embodiments, a nucleic acid construct of the invention encoding a CRISPR-Cas effector protein, a guide nucleic acid, reverse transcriptase, and/or a deaminase (e.g., a cytosine deaminase and/or adenine deaminase) may be introduced into a cell of an organism, thereby transforming the cell with the CRISPR-Cas effector protein, guide nucleic acid, reverse transcriptase, and/or deaminase. In some embodiments, a CRISPR-Cas effector protein and a guide nucleic acid may be introduced into a cell of an organism, optionally wherein the CRISPR-Cas effector protein and guide nucleic acid may be comprised in a complex (e.g., a ribonucleoprotein). In some embodiments, the organism is a eukaryote (e.g., a plant or mammal such as a human).

The term “transformation” as used herein refers to the introduction of a nucleic acid, polypeptide, and/or ribonucleoprotein (e.g., a heterologous nucleic acid, polypeptide, and/or ribonucleoprotein) into a cell. Transformation of a cell may be stable or transient. Thus, in some embodiments, a host cell or host organism may be stably transformed with a polynucleotide/nucleic acid molecule of the invention. In some embodiments, a host cell or host organism may be transiently transformed with a nucleic acid construct, a polypeptide, and/or a ribonucleoprotein of the invention.

“Transient transformation” in the context of a polynucleotide means that a polynucleotide is introduced into a cell (e.g., by a transformation and/or transfection approach) and does not integrate into the genome of the cell; thus, the cell is transiently transformed with the polynucleotide. A nucleic acid that is “transiently expressed” as used herein refers to a nucleic acid that has been introduced into a cell and the nucleic acid is not integrated into the genome of the cell, thereby the cell is transiently transformed with the nucleic acid.

By “stably introducing” or “stably introduced” in the context of a polynucleotide introduced into a cell (e.g., by a transformation and/or transfection approach) is intended that the introduced polynucleotide is stably incorporated into the genome of the cell, and thus the cell is stably transformed with the polynucleotide. A nucleic acid that is “stably expressed” as used herein refers to a nucleic acid that has been introduced into a cell and the nucleic acid is integrated into the genome of the cell, thereby the cell is stably transformed with the nucleic acid.

“Stable transformation” or “stably transformed” as used herein means that a nucleic acid molecule is introduced into a cell (e.g., by a transformation and/or transfection approach) and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a mammal, plant, etc.). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

A method of the present invention and/or blending step may process about 8, 9, 10, 11, 12, 13, 14, 15, or 16 ounces to about 17, 18, 19, 20, 21, 22, 23, 24, 25, 26, 27, 28, 29, 30, 31, or 32 ounces of plant material to provide wounded tissue in about 10 seconds or less such as about 9, 8, 7, 6, 5, 4, 3, 2, or 1 second(s). In some embodiments, a method of the present invention and/or blending step may process about 35 or 40 to about 45 or 50 clumps of plant material (e.g., plant shoots such about 6 week old plant shoots) to provide wounded tissue in about 10 seconds or less such as about 9, 8, 7, 6, 5, 4, 3, 2, or 1 second(s). In some embodiments, a method of the present invention and/or blending step may process about 35, 40, 45, or 50 clumps of plant material (e.g., plant shoots such about 6 week old plant shoots) to provide wounded tissue in about 10 seconds or less such as about 9, 8, 7, 6, 5, 4, 3, 2, or 1 second(s). In some embodiments, a method of the present invention and/or blending step may process about 5 grams to about 250 grams of plant material to provide wounded tissue in about 10 seconds or less such as about 9, 8, 7, 6, 5, 4, 3, 2, or 1 second(s).

In some embodiments, about 8, 9, 10, 11, 12, 13, 14, 15, or 16 ounces to about 17, 18, 19, 20, 21, 22, 23, 24, 25, 26, 27, 28, 29, 30, 31, or 32 ounces of plant material is blended with about 5, 6, 7, 8, 9, or 10 ounces to about 11, 12, 13, 14, 15, 16, 17, 18, 19, 20, 21, 22, 23, 24, 25, 26, 27, 28, 29, 30, 31, or 32 ounces of liquid (e.g., water and/or an aqueous composition optionally including a bacteria). In some embodiments, about 50 clumps of plant material (e.g., plant shoots such about 6 week old plant shoots) is blended with about 5, 6, 7, 8, 9, or 10 ounces to about 11, 12, 13, 14, 15, 16, 17, 18, 19, 20, 21, 22, 23, 24, 25, 26, 27, 28, 29, 30, 31, or 32 ounces of liquid (e.g., water and/or an aqueous composition optionally including a bacteria). In some embodiments, about 50 clumps of plant material (e.g., plant shoots such about 6 week old plant shoots) is blended with about 300 mL of liquid (e.g., water and/or an aqueous composition optionally including a bacteria). In some embodiments, about 5 grams to about 250 grams of plant material is blended with about 5, 6, 7, 8, 9, or 10 ounces to about 11, 12, 13, 14, 15, 16, 17, 18, 19, 20, 21, 22, 23, 24, 25, 26, 27, 28, 29, 30, 31, or 32 ounces of liquid (e.g., water and/or an aqueous composition optionally including a bacteria).

A method of the present invention may comprise a culturing step using methods known in the art. For example, culturing a plant material may include exposing a plant material, optionally a wounded plant tissue, to certain conditions (e.g., light, dark, nutrients, humidity, etc.) for a period of time (e.g., about 1, 2, 3, 4, or 5 day(s) to about 6, 7, 8, 9, 10, 11, 12, 13, 14, or more days). In some embodiments, culturing a plant material comprises providing temperature and/or light conditions sufficient to maintain and/or grow the plant material. In some embodiments, culturing a plant material is carried out before and/or after a blending step. In some embodiments, a method of the present invention comprises culturing a wounded plant tissue. A plant material and/or wounded tissue may be cultured in the presence of and/or on media that may include an antibiotic and/or a growth hormone (e.g., a plant growth hormone, and/or a nutrient).

In some embodiments, a method of the present invention comprises collects one or more node(s) from a plant (e.g., a blackberry, raspberry, or cherry plant) optionally a greenhouse grown plant, optionally sterilizing the node(s), cutting the sterilized node(s) by hand or blending in a blender to provide cut node tissue optionally having a smaller size than the sterilized node(s), and culturing the cut node tissue optionally on solid media for about 1 week to about 6 weeks or more. During the culturing step, the cut node tissue may produce multiple shoots (e.g., about 10-20 shoots per cut node). In some embodiments, a method of the present invention may comprise collecting the shoots and blending the shoots in a blender. In some embodiments, a callus is removed from the shoots prior to blending the shoots. In some embodiments, a callus is not removed from the shoots prior to blending the shoots and the shoots with the callus are blended. In some embodiments, about 10, 15, 20, or 25 clumps to about 30, 35, 40, 45, 50, 55, 60, 65, 70, 75, 80, 85, 90, 95, or 100 clumps of shoots (e.g., blackberry or cherry shoots) may be blended in a blender according to embodiments of the present invention. In some embodiments, in contrast to a method where certain plant material may be selected and/or isolated if wounded tissue is being provided by hand processing, in a method of the present invention this selection and/or isolation step is not performed and the desired plant material is blended with non-desired plant material. For example, in a method that is targeting cut stem tissue, a method providing the cut stem tissue by hand processing would cut off any leaves (e.g., non-desired plant material), but in a method of the present invention the entire stem with the leaves may be blended to provide the cut stem tissue.

Preparing wounded tissue by hand is a labor-intensive process. In some embodiments, a method of the present invention and/or step of preparing wounded tissue is devoid of hand manipulation (e.g., cutting plant tissue by hand) to result in the wounded tissue. In some embodiments, a method of the present invention and/or step of preparing wounded tissue is devoid of (e.g., does not involve) hand manipulation other than a step of providing (e.g., harvesting and/or collecting the plant tissue). In some embodiments, a method of the present invention and/or step of preparing wounded tissue is devoid of (e.g., does not involve) cutting plant tissue by hand to provide wounded plant tissue other than a step of providing (e.g., harvesting and/or collecting the plant tissue). For example, in some embodiments, a plant or plant part is collected, optionally by hand, to provide collected plant tissue and the collected plant tissue is wounded by a method that does not involve hand manipulation (e.g., cutting plant tissue by hand).

A method of the present invention may increase the amount of plant material prepared in a period of time compared to the amount of plant tissue prepared with a method that is devoid of blending using a blender (e.g., a method where the wounding consists of wounded tissue provided by hand processing and/or manipulation) in the same period of time for the same plant tissue. In some embodiments, a method of the present invention increases the amount of plant tissue prepared in a period of time compared to the amount of plant tissue prepared with a method that prepares wounded tissue by hand. For example, a method of the present invention may provide a volume of wounded plant tissue of about 8 ounces to about 32 ounces in about 5 seconds or less (e.g., about 1, 2, 3, 4 or 5 seconds), whereas a method of preparing wounded tissue that is devoid of blending using a blender prepares about 8 ounces to about 32 ounces in about 2 to about 4 hours. In some embodiments, a method of the present invention may wound about 50 clumps of plant material (e.g., plant shoots such about 6 week old plant shoots) in about 5 seconds or less (e.g., about 1, 2, 3, 4 or 5 seconds), whereas a method of preparing wounded tissue that is devoid of blending using a blender prepares about 50 clumps of plant material (e.g., plant shoots such about 6 week old plant shoots) in about 2 to about 4 hours. In some embodiments, a method of the present invention may provide wounded plant tissue in an amount of about 5 grams to about 250 grams in about 5 seconds or less (e.g., about 1, 2, 3, 4 or 5 seconds), whereas a method of preparing wounded tissue that is devoid of blending using a blender prepares about 5 grams to about 250 grams in about 2 to about 4 hours.

In some embodiments, a method of the present invention reduces tissue damage of a plant tissue (e.g., a wounded plant tissue) compared to a method of preparing the same plant tissue that is devoid of blending using a blender (e.g., a method where the wounding consists of wounded tissue provided by hand processing and/or manipulation). In some embodiments, a method of the present invention reduces tissue damage of a plant tissue compared to the amount of tissue damage provided by a method that prepares wounded tissue by hand. For example, a method of the present invention may reduce water soaking and/or browning of the wounded plant tissue. In some embodiments, wounded plant tissue prepared according to a method of the present invention has an increased survival rate and/or increased health compared to the survival rate and/or health of wounded plant tissue prepared according to a method that is devoid of blending using a blender (e.g., a method where the wounding consists of wounded tissue provided by hand processing and/or manipulation). A method of the present invention and/or wounded plant tissue prepared according to a method of the present invention may have a reduced death rate compared to the death rate of wounded plant tissue prepared according to a method that is devoid of blending using a blender (e.g., a method where the wounding consists of wounded tissue provided by hand processing and/or manipulation).

A method of the present invention and/or wounded plant tissue may provide an increased number of shoots that are propagated in vitro compared to the number of shoots that are propagated in vitro from plant tissue prepared using a method that is devoid of blending using a blender (e.g., a method where the wounding consists of wounded tissue provided by hand processing and/or manipulation).

A method of the present invention may provide plant tissue that has an increased transformation rate compared to the transformation rate of plant tissue prepared using a method that is devoid of blending using a blender (e.g., a method where the wounding consists of wounded tissue provided by hand processing and/or manipulation). For example, wounded tissue prepared according to a method of the present invention may have an increased transformation rate compared to the transformation rate for wounded tissue prepared using handing processing and/or manipulation.

A nucleic acid construct, polypeptide, and/or ribonucleoprotein of the invention can be introduced into a cell by any method known to those of skill in the art. In some embodiments, transformation methods include, but are not limited to, transformation via bacterial-mediated nucleic acid delivery (e.g., via Agrobacteria), viral-mediated nucleic acid delivery, silicon carbide and/or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the cell (e.g., a plant cell or an animal cell), including any combination thereof. In some embodiments of the invention, transformation of a cell comprises nuclear transformation. In some embodiments, transformation of a cell comprises plastid transformation (e.g., chloroplast transformation). In some embodiments, a recombinant nucleic acid construct of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

A nucleotide sequence, polypeptide, and/or ribonucleoprotein therefore can be introduced into a host organism or its cell in any number of ways that are well known in the art. The methods of the invention do not depend on a particular method for introducing one or more nucleotide sequence(s), polypeptide(s), and/or ribonucleoprotein(s) into the organism, only that they gain access to the interior of at least one cell of the organism. Where more than one nucleotide sequence, polypeptide, and/or ribonucleoprotein is to be introduced, they can be assembled as part of a single nucleic acid construct, or as separate nucleic acid constructs, and can be located on the same or different nucleic acid constructs. Accordingly, a nucleotide sequence, polypeptide, and/or ribonucleoprotein can be introduced into the cell of interest in a single transformation event, and/or in separate transformation events, or, alternatively, where relevant, a nucleotide sequence can be incorporated into a plant, for example, as part of a breeding protocol. In some embodiments, the cell is a eukaryotic cell (e.g., a plant cell or a mammalian such as a human cell).

Non-limiting examples of plants and/or parts thereof (e.g., explants) useful with the present invention include turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, Chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa, Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock. In some embodiments, a method and/or system of the present invention may be used in a method of modifying (e.g., editing) a plant and/or cell thereof (e.g., a plant and/or cell thereof from maize, soybean, wheat, canola, rice, tomato, pepper, sunflower, raspberry, blackberry, black raspberry and/or cherry). In some embodiments, the plant material is a blackberry, raspberry (e.g., red raspberry or black raspberry), artic bramble, or cherry plant material. In some embodiments, the plant material is from a plant in the Rubus family. In some embodiments, the plant material is from a plant in the Prunus family.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1: Wounding and Fragmentation

Approximately 50 clumps of in vitro blackberry material was combined with approximately 300 mL of liquid plant media and placed into a Cuisinart blender having model number CPB-300P1. After 1 pulse of the blender, the majority of the tissues were approximately 0.5 cm at the widest part of the tissue and while the majority of the tissues were wounded, there remained a significant number of tissues that were not wounded. After 2 pulses of the blender, fragments in the 0.2 to 0.4 cm (measured at the widest part of the tissue) range became more prevalent and nearly all of the tissue was wounded. After 3 pulses of the blender, fragments in the 0.2 to 0.4 cm (measured at the widest part of the tissue) range were the majority of the tissue sizes in the mixture. It was determined that for the purpose of generating wounded tissue for transformation, 2 pulses was sufficient. It was also determined that for the purpose of developing a callus screening system using the same starting tissue, the 3 pulse process was more advantageous as the smaller fragments lead to extensive callus growth.

### Example 2: Plant Tissue Damage

Using a blender, in vitro grown cherry plant explants were blended in water using a ratio of approximately 50 cherry shoots and 300 ml of water. The blended cherry tissue was removed from the water and placed onto a solid media surface and compared to tissue that was cut by hand and also placed on a solid media surface. Over the course of the next several weeks it was observed that the explants from the blender showed no browning while the explants cut by hand showed browning from the cut and browning where the explant was handled with forceps. Upon repeats of this experiment, it was observed that browning doesn't always occur in the hand cut tissues. However, if there is tearing or ripping of the tissue such as due to the dullness of a scalpel for the hand cut tissues, there will be browning.

A second experiment was performed essentially as described in the above paragraph except that, for the blended cherry tissue, the liquid in the blender also contained a suspension of Agrobacterium tumefaciens and, for the hand cut cherry tissue, the wounded explants were contacted with the suspension of Agrobacterium tumefaciens prior to placing the wounded explants on the solid media. The second experiment was a transformation experiment where the Agrobacterium tumefaciens was expected to transfer DNA to some of the cherry plant cells in the explant. A comparison of explants blended with the Agrobacterium solution and hand cut explants inoculated with the Agrobacterium solution also demonstrated that there was no browning of the explants from the blender method whereas there was browning of some of the hand-cut explants.

### Example 3: Transformation

In vitro grown cherry shoots were blended in an Agrobacterium tumefaciens inoculum solution containing Agrobacterium resuspended in plant-growth medium. Blended explants were plated and evenly spread on semi-solid medium for co-culture. The Agrobacterium strain contained a transformation plasmid which included the fluorescent marker, ZsGreen. To determine transformation efficiency, the blender-inoculated explants were evaluated for fluorescence and it was observed that ˜90% of explants were highly fluorescing after one week. Eventually, transgenic fluorescing callus tissue formed. A similar experiment was performed as described above in Example 2 except the blender was replaced with hand cutting of cherry shoots to provide wounded tissue and subsequently contacting the wounded tissue with a suspension of Agrobacterium tumefaciens. In the hand cut experiment, approximately 10% of explants were fluorescing which was considerably less when compared to blender-processed explants.

### Example 4: Regeneration and Propagation in Blackberry

Leaves and petioles from approximately 50 clumps of in vitro propagated blackberry shoots were excised using sterile scissors in a laminar flow hood. Excised tissues were placed into approximately 300 mL of sterile water in a Cuisinart blender model CPB-300P1 before being blended using 2 pulses, strained, and then blotted until dry using sterile paper towels. The dry tissues were distributed evenly into plates containing a suitable shoot induction medium. After 3-6 weeks of growth, de novo shoots could be observed regenerating from the blended leaf and petiole tissues. Shoots grew to about 1-1.5 cm tall after several more weeks of culture and were then excised and rooted in a suitable rooting medium over a period of 2-3 weeks to produce plantlets that were transplanted to growth medium and transitioned to the greenhouse.

Similar to the above method, whole shoots were blended and cultured to further propagate shoots in vitro. Shoots were blended, blotted and distributed as described in the paragraph above but were placed onto a suitable propagation medium. The fragmented tissues containing nodes regenerated after 3-6 weeks of culture in the form of small shoot clusters that were further utilized in sterile culture.

1. A method of preparing a plant tissue for propagation, regeneration, and/or transformation, the method comprising:
wounding a plant or plant part to provide a wounded tissue, wherein the wounding comprises blending the plant or plant part in a blender, wherein the blender comprises a single blade, thereby providing the plant tissue.

2. The method of claim 1, wherein the blending comprises blending a whole plant.

3. The method of claim 1, wherein the blending comprises blending a leaf, node, shoot, and/or callus.

4. The method of claim 1, wherein the method increases the amount of wounded tissue prepared in a period of time compared to the amount of wounded tissue prepared with a method that is devoid of blending using a blender in the same period of time for the same plant or plant part.

5. The method of claim 1, wherein the single blade is a chopping blade.

6. The method of claim 1, wherein the blending comprises blending the plant or plant part at about 1 Hz to about 380 Hz for at least about 1 second.

7. The method of claim 1, wherein the blending provides at least about 8 ounces to about 32 ounces or about 50 clumps of wounded tissue in about 1 second, to about 5 seconds.

8. The method of claim 1, wherein the method is devoid of hand manipulation to prepare the wounded tissue.

9. The method of claim 1, wherein the wounding step consists of blending the plant or plant part in the blender.

10. The method of claim 1, wherein the method reduces tissue damage of the plant tissue compared to a method of preparing the same plant tissue that is devoid of blending using a blender.

11. The method of claim 1, wherein the plant tissue has an increased transformation rate compared to a method of preparing the same plant tissue that is devoid of blending using a blender.

12. The method of claim 1, wherein the plant tissue provides an increased number of shoots that are propagated in vitro compared to a method of preparing the same plant tissue that is devoid of blending using a blender.

13. The method of claim 1, wherein the method has a reduced death rate of the plant tissue compared to the death rate of plant tissue prepared with a method that is devoid of blending using a blender for the same plant or plant part.

14. The method of claim 1, wherein the plant or plant part is a Rubus plant or plant part.

15. The method of claim 1, further comprising culturing the plant tissue on and/or in a growth media.

16. The method of claim 1, further comprising prior to wounding the plant or plant part, collecting the plant part.

17. The method of claim 1, wherein blending the plant or plant part in the blender comprises blending in the blender the plant or plant part in the presence of water and/or an aqueous composition.

18. The method of claim 1, wherein blending the plant or plant part in the blender comprises blending in the blender the plant or plant part in the presence of a bacteria.

19. The method of claim 1, wherein blending the plant or plant part in the blender comprises blending in the blender the plant or plant part in the presence of a bacteria and the method further comprises introducing a polynucleotide from the bacteria into a cell of the plant or plant part.

20. The method of claim 19, further comprising culturing the wounded tissue.
